(19) United States
(12) Patent Application Publication
(10) Pub. No.: US 2011/0159084 A1
(43) Pub. Date: Jun. 30, 2011

(54) RALOXIFENE PHARMACEUTICAL FORMULATIONS

(75) Inventors: Alagumurugan Alagarsamy, Tuticorin District (IN); Boorugu Rambabu, Nalgonda District (IN); Pallempalli Siva Reddy, Hyderabad (IN); Kumaran Venugopal, Chennai (IN); Bhasin Rakesh Kumar, Ghaziabad (IN)

(73) Assignees: DR. REDDY'S LABORATORIES LTD., Andhra Pradesh (IN); DR. REDDY'S LABORATORIES, INC., Bridgewater, NJ (US)

(21) Appl. No.: 12/936,804

(22) PCT Filed: Apr. 2, 2009
(86) PCT No.: PCT/US2009/039273

§ 371 Date: Oct. 7, 2010

Related U.S. Application Data
(60) Provisional application No. 61/075,507, filed on Jun. 25, 2008.

(30) Foreign Application Priority Data
Apr. 2, 2008 (IN) 834/CHE/2008

(51) Int. Cl. A61K 9/48; A61K 31/4535; A61P 19/10; A61K 9/14

(52) U.S. Cl. 424/452; 514/324; 424/400

(57) ABSTRACT

Pharmaceutical formulations comprising raloxifene or its salts, esters, polymorphs, isomers, hydrates, solvates, or derivatives thereof having defined particle sizes. Also described are processes for preparing formulations and methods of using such formulations.

### INTRODUCTION

An aspect of the present invention relates to pharmaceutical formulations comprising raloxifene, including any of its salts, esters, polymorphs, isomers, hydrates, solvates, and derivatives. The present invention also provides processes for preparing raloxifene-containing formulations and methods of using such formulations.

Further, the present invention relates to pharmaceutical formulations comprising raloxifene, including its salts, esters, polymorphs, isomers, hydrates, solvates and derivatives, having defined particle sizes.

Raloxifene is an estrogen agonist/antagonist, commonly referred to as a selective estrogen receptor modulator (SERM), which belongs to the benzothiophene class of compounds. Raloxifene is represented by structural formula (1).

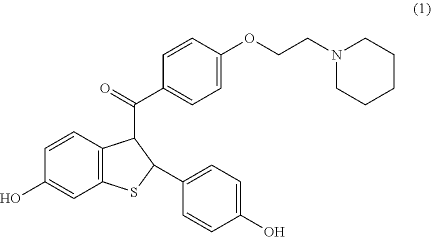

A chemical name for raloxifene hydrochloride is methanone, [6-hydroxy-2-(4-hydroxyphenyl)benzo[b]thiene-3-yl]-[4-[2-(1-piperidinyl)ethoxy]phenyl]-, hydrochloride. Raloxifene hydrochloride has the empirical formula C₂₈H₂₇NO₄S.HCl, corresponding to a molecular weight of 510.05. Raloxifene hydrochloride is an off-white to pale yellow solid that is very slightly soluble in water, the water solubility being approximately 0.3 g/ml at 25° C., and significantly lower in simulated gastric fluid (SGF) USP (0.003 mg/ml) and simulated intestinal fluid (SIF) USP (0.002 mg/ml), at 37° C.

Raloxifene hydrochloride is available in oral tablets, sold by Eli Lilly and Company using the brand EVISTA®. Each EVISTA tablet contains 60 mg of raloxifene hydrochloride, which is the molar equivalent of 55.71 mg of raloxifene. Inactive ingredients in the tablets include anhydrous lactose, carnauba wax, crospovidone, FD&C Blue No. 2 aluminium lake, hypromellose, lactose monohydrate, magnesium stearate, modified pharmaceutical glaze, polyethylene glycol, polysorbate 80, povidone, propylene glycol, and titanium dioxide. EVISTA is prescribed for the treatment and prevention of osteoporosis in postmenopausal women.

Absolute bioavailability of raloxifene is only 2%, and this may be attributed to extensive first pass metabolism in the gut and liver to form the glucoronide conjugates: raloxifene-4′-glucoronide; raloxifene-6-glucoronide; raloxifene-6,4′-diglucoronide; etc. Raloxifene exhibits high intra-subject pharmacokinetic variability (approximately 30% coefficient of variation).

Osteoporosis is a disease of bone that leads to an increased risk of fracture. In osteoporosis, the bone mineral density (BMD) is reduced, bone microarchitecture is disrupted, and the amount and variety of non-collagenous proteins in bone is altered. Osteoporosis is most common in women after menopause, when it is called postmenopausal osteoporosis, but may also develop in elderly men, and may occur in anyone in the presence of particular hormonal disorders and other chronic diseases or as a result of medications, specifically glucocorticoids, when the disease is called steroid- or glucocorticoid-induced osteoporosis (SIOP or GIOP). Given its influence on the risk of fragility fractures, osteoporosis may significantly affect life expectancy and quality of life.

Bone loss is common in postmenopausal women. Raloxifene works like the hormone estrogen to slow this bone loss and can even help increase normal bone growth. Thus raloxifene decreases resorption of bone and reduces biochemical markers of bone turnover to the premenopausal range. Estrogen can cause side effects like vaginal bleeding and breast tenderness. It can also increase the risk of breast or uterine cancer. Raloxifene doesn't have these same side effects. In addition, raloxifene also lowers total and low-density lipoprotein (LDL) cholesterol (also known as “bad” cholesterol) but doesn't raise high-density lipoprotein (HDL) cholesterol (also known as “good” cholesterol).

Raloxifene and its derivatives as anti-estrogenic or anti-androgenic compounds are disclosed in U.S. Pat. No. 4,418,068.

As it is known from the literature that raloxifene hydrochloride is insoluble in water and has only about 2% absolute bioavailability, attempts have been made to improve in vitro dissolution as well as in vivo bioavailability.

U.S. Pat. Nos. 5,393,763, 5,457,117, 5,478,847, and 6,906,086, and U.S. Reissue Pa. Nos. RE 38,968 and RE 39,049, disclose methods of use of benzothiophenes for inhibiting bone loss. U.S. Pat. Nos. 5,972,383, 6,458,811, 5,811,120, 6,797,719, and 6,894,064 disclose solid oral formulations containing benzothiophenes.

U.S. Pat. Nos. 5,811,120 and 5,972,383 disclose solid oral compositions comprising raloxifene in combination with a surfactant, polyvinylpyrrolidone, and a water soluble diluent, wherein surfactants comprise sorbitan fatty acid esters or polyoxyethylene sorbitan fatty acid esters, and the water soluble diluent is a polyol or a sugar.

U.S. Pat. No. 6,458,811 discloses pharmaceutically acceptable salts and solvates of raloxifene characterized in that the compound is in particulate form and has a specific size range, said particles having a mean particle size less than about 25 microns, and at least about 90% of said particles having sizes less than about 50 microns. It is said that the particle size distribution range claimed in this patent will provide consistent in vivo absorption/bioavailability characteristics.

U.S. Pat. Nos. 6,797,719 and 6,894,064 disclose solid oral pharmaceutical compositions comprising solvents and solvates of raloxifene, characterized in that the compound is in particulate form, said particles having a mean particle size less than about 25 microns, and at least about 90% of said particles having sizes less than about 50 microns, a surfactant, and a water-soluble diluent. In order to achieve a consistent in vitro dissolution profile and in vivo bioavailability, raloxifene hydrochloride is preferably micronized.

Many approaches can be tried to improve the solubility and dissolution of poorly soluble active ingredients. Some of the approaches include salt formation, particle size reduction, pH adjustment, use of surfactants, inclusion complexes with cyclodextrins, formation of co-precipitates with hydrophilic polymers, and co-milling with hydrophilic excipients, to name a few.

The compositions of raloxifene disclosed in the art mainly focus on utilization of micronized raloxifene to improve the solubility/bioavailability of the active agent. Processes such as milling or grinding of the active substance have been employed in order to obtain finer particles having an increased surface area to improve the bioavailability. The active substance with increased surface area is further formulated along with water soluble fillers and a surfactant to improve the bioavailability of the raloxifene. The milling/micronization processes generally employed to reduce particle size of raloxifene are cumbersome, require specialized machinery, involve loss of the active agent during the size reduction process, and also involve additional cost. Further, milled/micronized raloxifene essentially in the form of very finely divided material presents difficulties during processing into a dosage form such as a tablet preparation or during capsule filling, since such micronized material tend to agglomerate and exhibits poor flow properties. Such processing difficulties, particularly the poor flow properties, invariably lead to non-homogeneity in the dosage form, content uniformity issues and unacceptable batch-to-batch variability, which are highly undesirable. Additionally, the milling process generates heat and pressure on the active agent particles, which can lead to alteration in the physicochemical properties of the drug such as degradation of the compound; thus such milling techniques should be avoided.

There is always a dynamic between the properties which yield a maximum bioavailability (increased particle surface area) and the practical limits of manufacture. The point of compromise which marks this “best solution” is unique to each situation and unique as to its determination.

Because of its poor solubility and poor bioavailability, there exists a need for developing improved formulations containing raloxifene which are stable and possess desired bioavailability. There is a need for improved formulations comprising unmicronized/unmilled raloxifene or its salts having desirable bioavailability characteristics.

### SUMMARY

The present invention relates to pharmaceutical formulations comprising raloxifene, including its salts, esters, polymorphs, isomers, hydrates, solvates and derivatives.

In an embodiment, the invention relates to pharmaceutical formulations wherein raloxifene, including its salts, esters, polymorphs, isomers, hydrates, solvates and derivatives, has defined particle size distributions.

In an aspect, the invention relates to pharmaceutical formulations made using defined particle size distributions of raloxifene, including its salts, esters, polymorphs, isomers, hydrates, solvates and derivatives, wherein the mean particle size is in the range of about 30 μm to about 150 μm, and D₉₀ is a value in the range of about 60 μm to about 500 μm.

An aspect of the invention provides pharmaceutical formulations comprising raloxifene, including its salts, esters, polymorphs, isomers, hydrates, solvates and derivatives, as an active agent, at least one surfactant and at least one water insoluble diluent, wherein the mean particle size of the active agent is about 30 μm to about 75 μm.

An aspect of the invention provides pharmaceutical formulations comprising unmicronized raloxifene, including its salts, esters, polymorphs, isomers, hydrates, solvates and derivatives, as an active agent, at least one surfactant, and optionally at least one water insoluble diluent, wherein the mean particle size of the active agent is about 30 μm to about 75 μm.

In an embodiment, the invention includes pharmaceutical formulations comprising unmicronized raloxifene hydrochloride and at least one surfactant, wherein the raloxifene hydrochloride and surfactant are in intimate contact with each other.

An aspect of the invention relates to pharmaceutical formulations comprising raloxifene, including its salts, esters, polymorphs, isomers, hydrates, solvates and derivatives, and at least one surfactant, wherein weight ratios of raloxifene to surfactant is in the range of about 0.3 to about 10, or about 0.4 to about 5, or about 0.5 to about 2.

In an embodiment, the invention relates to pharmaceutical formulations wherein raloxifene, including its salts, esters, polymorphs, isomers, hydrates, solvates and derivatives, has defined particle sizes, and weight ratios of raloxifene to surfactant is in the range of about 0.3 to about 10, or about 0.4 to about 5.

In an embodiment, the pharmaceutical formulations of the present invention comprise unmicronized raloxifene or a pharmaceutically acceptable salt as an active agent, at least one non-ionic surfactant which is in intimate contact with the active substance, at least one water insoluble diluent, optionally together with one or more pharmaceutically acceptable excipients, wherein the mean particle size of the active agent is about 30 μm to about 75 μm.

An aspect of the invention provides pharmaceutical formulations comprising raloxifene, including its salts, esters, polymorphs, isomers, hydrates, solvates and derivatives, as an active agent, at least one surfactant, and optionally at least one water insoluble diluent, wherein D₉₀ of the particles of the active agent is a value between about 60 μm and about 150 μm.

An aspect of the invention provides pharmaceutical formulations comprising raloxifene, including its salts, esters, polymorphs, isomers, hydrates, solvates and derivatives, as an active agent; at least one surfactant, and optionally at least one water insoluble diluent, wherein the mean particle size of the active agent is about 30 μm to about 75 μm and D₉₀ of the particles of the active agent is a value between about 60 μm and about 150 μm.

In an embodiment, the invention relates to pharmaceutical formulations comprising raloxifene hydrochloride as an active agent, at least one surfactant, and optionally at least one water insoluble diluent, wherein raloxifene hydrochloride particles have a defined particle size distribution such that the mean particle size of raloxifene hydrochloride is in the range of about 30 μm to about 75 μm and the D₉₀ is a value in the range of about 60 μm to about 150 μm, and wherein weight ratios of raloxifene hydrochloride to surfactant are in the range of about 0.3 to about 3, or about 0.5 to about 2.

In an embodiment, the present invention provides formulations that exhibit excellent stability during storage.

In an embodiment, the pharmaceutical formulations of the present invention are substantially free of N-oxide impurity.

In an embodiment, the pharmaceutical formulations of the present invention contain less than about 2%, or less than about 1%, or less than about 0.5%, by weight of total impurities, expressed as a percentage of the raloxifene content of the formulation.

In an embodiment, the pharmaceutical formulations of the present invention include one or more antioxidants.

Further, the invention relates to processes for preparing pharmaceutical formulations.

An embodiment of the invention includes processes to prepare formulations wherein the process comprises a step wherein raloxifene hydrochloride is dispersed in a granulating fluid, which is further processed to obtain a desired formulation.

The invention also relates to methods of using pharmaceutical formulations for preventing and/or treating osteoporosis in a subject in need thereof, such as in postmenopausal women.

### DETAILED DESCRIPTION

The present invention provides pharmaceutical formulations comprising raloxifene, including its salts, esters, polymorphs, isomers, hydrates, solvates and derivatives. The invention also provides pharmaceutical formulations wherein raloxifene, including its salts, esters, polymorphs, isomers, hydrates, solvates and derivatives, has defined particle size distributions.

In an embodiment the invention relates to pharmaceutical formulations wherein the active agent raloxifene, including any of its salts, esters, polymorphs, isomers, hydrates, solvates and derivatives, is present in an amorphous form, crystalline form, or mixtures thereof.

In an embodiment the invention relates to pharmaceutical formulations wherein the raloxifene hydrochloride is present as an active agent together with at least one pharmaceutically acceptable excipient, as a solid dispersion, such as a co-precipitate or a solid solution.

A solid dispersion is defined as a dispersion of one or more active agents in an inert carrier or matrix in a solid state, prepared by melting (fusion), solvent, or melting-solvent methods. Dispersions obtained through a fusion process are often called melts, and those obtained by a solvent method are frequently referred to as co-precipitates or co-evaporates. Chiou and Riegelman classified solid dispersions into six representative types: simple eutectic mixtures, solid solutions, glass solutions and glass suspensions, amorphous precipitates in a crystalline carrier, compound or complex formation, and combinations thereof. In a solid solution, the components form a homogeneous one-phase system.

The pharmaceutically acceptable salts can exist in conjunction with raloxifene, its esters, or its ethers as acid addition salts, primary, secondary, tertiary, or quaternary ammonium, alkali metal, or alkaline earth metal salts.

Acids commonly employed to form acid addition salts include inorganic acids such as hydrochloric, hydrobromic, hydriodic, sulfuric, and phosphoric acids, as well as organic acids such as toluenesulfonic, methanesulfonic, oxalic, p-bromophenylsulfonic, carbonic, succinic, citric, benzoic, and acetic acids, and related inorganic and organic acids. Such pharmaceutically acceptable salts of these and other acids include the sulfate, pyrosulfate, bisulfate, sulfite, bisulfite, phosphate, ammonium, monohydrogen phosphate, dihydrogen phosphate, meta-phosphate, pyrophosphate, chloride, bromide, iodide, acetate, propionate, decanoate, caprolate, acrylate, formate, isobutyrate, caprate, heptanoate, propionate, oxalate, malonate, succinate, subarate, sebacate, fumarate, hippurate, maleate, butyne-1,4-dioate, hexyne-1,6-dioate, benzoate, chlorobenzoate, methyl benzoate, dinitrobenzoate, hydroxybenzoate, methoxybenzoate, phthalate, sulfonate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, and mandelate salts. Also included are the ammonium, magnesium, tetramethylammonium, potassium, trimethylammonium, sodium, methylammonium, calcium, and other salts.

Recitation of “raloxifene” herein is intended to include the compound known as raloxifene, as well as any substance that provides raloxifene to the body following administration of a pharmaceutical dosage form.

In an aspect, the invention provides pharmaceutical formulations made using defined particle size distributions of raloxifene, including its salts, esters, polymorphs, isomers, hydrates, solvates and derivatives, wherein the mean particle size is in the range of about 30 μm to about 150 μm, and D₉₀ is a value in the range of about 60 μm to about 500 μm. In an aspect, the active agent is unmicronized.

The term “unmicronized” or “unmilled” according to the present invention implies that no specific or distinct technique, with the intention of reducing particle sizes, such as milling or micronization, is applied to the active agent.

The term “mean particle size” for purposes of the present invention is defined as an average of the sizes of all the particles in a powder.

In an embodiment, the present invention provides pharmaceutical formulations comprising raloxifene, including its salts, esters, polymorphs, isomers, hydrates, solvates and derivatives, as an active agent, at least one surfactant and at least one water insoluble diluent. In an aspect, the mean particle size of the active agent is about 30 μm to about 75 μm.

An aspect of the invention provides pharmaceutical formulations comprising unmicronized raloxifene hydrochloride particles as an active agent, at least one surfactant, and optionally at least one water insoluble diluent. In a further aspect, the mean particle size of the said active agent is about 30 μm to about 75 μm.

In an embodiment, the invention provides pharmaceutical formulations comprising unmicronized raloxifene hydrochloride and at least one surfactant, wherein the raloxifene hydrochloride and surfactant are in intimate contact with each other.

An aspect of the invention provides pharmaceutical formulations comprising raloxifene, including its salts, esters, polymorphs, isomers, hydrates, solvates and derivatives, and at least one surfactant, wherein weight ratios of active agent to surfactant are in the range of about 0.3 to about 10, or about 0.4 to about 5, or about 0.5 to about 2.

In an embodiment, the invention provides pharmaceutical formulations wherein raloxifene, including its salts, esters, polymorphs, isomers, hydrates, solvates and derivatives, has defined particle sizes, and weight ratios of raloxifene or its salts, esters, polymorphs, isomers, hydrates, solvates or derivatives to surfactant are in the range of about 0.3 to about 10, or about 0.4 to about 5.

In an embodiment, pharmaceutical formulations of the present invention comprise unmicronized raloxifene or a pharmaceutically acceptable salt as an active agent, at least one non-ionic surfactant which is in intimate contact with the active substance, at least one water insoluble diluent, and optionally one or more pharmaceutically acceptable excipients. In an aspect, the mean particle size of the active agent is about 30 μm to about 75 μm.

The term “intimate contact” for aspects of the present invention can include the contact of particles obtained from rigorous mixing of powders. However, an intimate contact according to an aspect of the present invention is obtained by dispersing the raloxifene salts or solvates in a surfactant solution, or by thorough mixing of active agent and the surfactant using appropriate mixing techniques, such as wet granulation, or dry granulation.

An aspect of the invention provides pharmaceutical formulations comprising a raloxifene active agent, at least one surfactant, and optionally at least one water insoluble diluent, wherein D₉₀ of the particles of the active agent is a value between about 60 μm and about 150 μm.

An aspect of the invention provides pharmaceutical formulations comprising raloxifene, including its salts, esters, polymorphs, isomers, hydrates, solvates and derivatives, as an active agent, at least one surfactant, and optionally at least one water insoluble diluent, wherein the mean particle size of the active agent is about 30 μm to about 75 μm and the D₉₀ of the active agent is a value between about 60 μm and about 150 μm.

In an embodiment, the invention provides pharmaceutical formulations comprising raloxifene hydrochloride, at least one surfactant, and optionally at least one water insoluble diluent, wherein raloxifene hydrochloride particles have a defined particle size distribution such that a mean particle size of raloxifene hydrochloride is in the range of about 30 μm to about 75 μm and D₉₀ is a value in the range of about 60 μm to about 150 μm, and wherein weight ratios of raloxifene hydrochloride to the surfactant are in the range of about 0.3 to about 3, or about 0.5 to about 2.

Aqueous solubility of raloxifene hydrochloride is far lower than would be expected for an organic hydrochloride salt. This poor solubility has somewhat limited the bioavailability of this salt form. Another significant barrier to optimum and consistent absorption of raloxifene hydrochloride is its hydrophobicity.

Surfactants are wetting agents that lower the surface tension of a liquid, allowing easier spreading, and lower the interfacial tension between two liquids. Thus they have been used to increase the solubility of some poorly soluble drugs.

It has been observed that the use of defined ratios of raloxifene hydrochloride to surfactant permits making formulations that have bioavailability similar to that of the commercial product, i.e., EVISTA tablets.

Defined weight ratios of raloxifene hydrochloride to surfactant in the present invention are in the range of about 0.3 to about 10, or about 0.4 to about 5 or about 0.5 to about 2.

A surfactant can be classified by the presence and type of charged groups in its head. The head of an ionic surfactant carries a net charge. If the charge is negative, the surfactant is more specifically called anionic; if the charge is positive, it is called cationic. If a surfactant contains a head with two oppositely charged groups, it is termed zwitterionic. A nonionic surfactant has no charge groups in its head. Most preferrably the surfactants are selected from the group of non-ionic surfactants.

Non-limiting examples of anionic surfactants include the alkoyl isethionates, alkyl and alkyl ether sulfates and salts thereof, alkyl and alkyl ether phosphates and salts thereof, alkyl methyl taurates, and soaps, such as, for example, alkali metal salts including sodium or potassium salts of long chain fatty acids.

Various amphoteric and zwitterionic surfactants are those which are broadly described as derivatives of aliphatic secondary and tertiary amines, in which the aliphatic radical can be straight or branched chain and wherein one of the aliphatic substituents contains from about 8 to about 22 carbon atoms and one contains an anionic water solubilizing group, e.g., carboxy, sulfonate, sulfate, phosphate, or phosphonate. Examples are alkyliminoacetates, iminodialkanoates, aminoalkanoates, imidazolinium and ammonium derivatives. Other suitable amphoteric and zwitterionic surfactants include betaines, sultaines, hydroxysultaines, alkylsarcosinates and alkanoylsarcosinates.

The silicone surfactants are typically organically modified organopolysiloxanes. Useful silicone surfactants include dimethicone copolyols. These materials are polydimethyl siloxanes, which have been modified to include polyether side chains such as polyethylene oxide chains, polypropylene oxide chains, mixtures of these chains, and polyether chains containing moieties derived from both ethylene oxide and propylene oxide.

Non-ionic surfactants include those that can be broadly defined as condensation products of long chain alcohols, e.g., O₈₋₃₀ alcohols, with sugar or starch polymers, i.e., glycosides. Various sugars include but are not limited to glucose, fructose, mannose, and galactose; and various long chain alcohols include but are not limited to decyl alcohol, cetyl alcohol, stearyl alcohol, lauryl alcohol, myristyl alcohol, oleyl alcohol, and the like. Commercially available examples of this type of surfactants include decylpolyglucoside (available as APG 325 CS from Henkel) and laurylpolyglucoside (available as APG 600 CS and 625 CS from Henkel).

Other useful non-ionic surfactants include alkylpolyethylene oxides, copolymers having poly(ethylene oxide) and poly(propylene oxide) chains and called “poloxamers,” poloxamines, and the condensation products of alkylene oxides with fatty acids (i.e., alkylene oxide esters of fatty acids). Other non-ionic surfactants are the condensation products of alkylene oxides with 2 moles of fatty acids (i.e., alkylene oxide diesters of fatty acids). Other non-ionic surfactants are the condensation products of alkylene oxides with fatty alcohols (i.e., alkylene oxide ethers of fatty alcohols). Still other non-ionic surfactants are the condensation products of alkylene oxides with both fatty acids and fatty alcohols [i.e., wherein the polyalkylene oxide portion is esterified on one end with a fatty acid and etherified (i.e. connected via an ether linkage) on the other end with a fatty alcohol]. Non-limiting examples of these alkylene oxide derived non-ionic surfactants include ceteth-6, ceteth-10, ceteth-12, ceteareth-6, ceteareth-10, ceteareth-12, steareth-6, steareth-10, steareth-12, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10. Other non-ionic surfactants include sugar esters and polyesters, alkoxylated sugar esters and polyesters, C₁₋₃₀ fatty acid esters of C₁₋₃₀ fatty alcohols, alkoxylated ethers of C₁₋₃₀ fatty alcohols, polyglyceryl esters of C₁₋₃₀ fatty acids, C₁₋₃₀ esters of polyols, C₁₋₃₀ ethers of polyols, alkyl phosphates, polyoxyalkylene fatty ether phosphates, fatty acid amides, acyl lactylates, and mixtures thereof. Non-limiting examples of these surfactants include: polyethylene glycol 20 sorbitan monolaurate (polysorbate 20), polyethylene glycol 5 soya sterol, steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof. Further examples of suitable emulsifiers include mixtures of stearyl octanoate and isopropyl myristate, or mixtures of cetyl octanoate and stearyl octanoate.

In an embodiment, the invention includes surfactants which have significant P-gp (para-glycoprotein) inhibitory activity such as copolymers of poly(ethylene oxide) and poly(propylene oxide), polysorbate 80, polyglycolized glycerides such as are available commercially in products from Gattefossé such as GELUCIRE™ 40/14, GELUCIRE 42/12, and GELUCIRE 50/13, Vitamin E TPGS, and the like.

In a further embodiment the invention includes poloxamers or Vitamin E TPGS or combinations thereof, used as surfactants.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist those have slightly different properties. Different grades, such as poloxamer 188 and poloxamer 407, are available. A poloxamer 407 product is commercially available as Pluronic™ F 127 from BASF, and has a central block of poly(propylene oxide) of about 56 repeat units, flanked by two poly(ethylene oxide) blocks, each having about 101 repeat units.

In some embodiments, cyclodextrins or their derivatives may be used to enhance solubility. Various cyclodextrins or derivatives include but are not limited to the natural cyclodextrins including α-cyclodextrin, β-cyclodextrin and Γ-cyclodextrin. Any of the natural cyclodextrins can be derivatized, such as derivatives of β-cyclodextrin. Cyclodextrin derivatives include alkylated cyclodextrins, comprising methyl-, dimethyl-, trimethyl- and ethyl-β-cyclodextrins; hydroxyalkylated cyclodextrins, including hydroxyethyl-, hydroxypropyl-, and dihydroxypropyl-β-cyclodextrin; ethyl carboxymethyl cyclodextrins; sulfate, sulfonate and sulfoalkyl cyclodextrins, such as β-cyclodextrin sulfate, β-cyclodextrin sulfonate, and β-cyclodextrin sulfobutyl ether; as well as polymeric cyclodextrins. Other cyclodextrin derivatives can be made by substitution of the hydroxy groups with saccharides, such as glucosyl- and maltosyl-β-cyclodextrin.

Co-precipitates of raloxifene hydrochloride with hydrophilic materials also can enhance its solubility.

The percentage of particles with different dimensions that exist in a powder is called the particle size distribution. It is represented in certain ways. Particle size is the maximum dimension of a particle, frequently expressed in units of μm. Particle size distributions can be expressed in terms of, D₁₀, D₅₀, D₉₀ and D_{[4,3]}. The D₁₀, D₅₀ and D₉₀ represent the 10th, median or the 50th percentile, and the 90th percentiles of the particle size distribution, respectively, as measured by volume. That is, the D₁₀, D₅₀, D₉₀ is a value of the distribution such that 10%, 50%, 90% by volume of the particles have a size of this value or less, or is the percentage of particles smaller than that size. D₅₀ is also known as median diameter of particle. It is one of the important parameters representing characteristics of particle of powder. For a sample, if D₅₀=5 μm, it means that 50% of the particles are smaller than 5 μm. Similarly, if D₁₀=5 μm, 10% by volume of the particles are less than or equal to 5 μm, and if D₉₀=5 μm, 90% of the particles are less than or equal to 5 μm. D_{[4,3]} is the volume moment mean of the particle or the volume weighted particle size.

Equipment using a laser diffraction principle such as a Malvern Particle Size Analyzer (Malvern Instruments Ltd., Malvern, Worcestershire, United Kingdom) or Horiba Laser Scattering Particle Size Distribution Analyzer (Horiba Ltd., Kyoto, Japan) may be used for determining particle size distributions. Other techniques and equipment known in the art may also be used for particle size measurements.

In an embodiment, the invention includes formulations prepared using unmicronized raloxifene hydrochloride of defined particle size distribution, wherein mean particle sizes are in the range of about 30 μm to about 150 μm, or about 30 μm to about 75 μm. D₉₀ is a value in the range of about 60 μm to about 500 μm, or about 60 μm to about 150 μm.

In an embodiment the invention relates to pharmaceutical formulations prepared using raloxifene hydrochloride having any one or more of: mean particle sizes in the range of about 30 μm to about 150 μm, or about 30 μm to about 75 μm; D₉₀ in the range of about 60 to about 150 μm; and weight ratios of raloxifene hydrochloride to surfactant in the range of about 0.4 to about 3, or about 0.5 to about 2.

In addition to improvement in the solubility of the active in the pharmaceutical formulation, it is equally important that the pharmaceutical formulations are adequately stable during storage over their commercial shelf life. Formation of an N-oxide impurity, due to interaction of raloxifene with peroxides, is reported in the literature. The peroxides are commonly present in excipients like crospovidone and povidone. The peroxide impurities in the excipients containing peroxides could promote oxidation of the drug substance by direct oxidation of tertiary amine by peroxide. The formation of an N-oxide impurity in raloxifene formulation is considered to be due to direct oxidation of the tertiary amine by the peroxides present in excipients.

In an embodiment, the pharmaceutical formulations of the present invention are substantially free of N-oxide impurity, even when one or more excipients containing peroxide impurities are used in the formulation. In an aspect, the excipients containing peroxide impurities such as povidone or crospovidone may be used to make the formulations of the present invention.

Oxidation reactions can be prevented by use of antioxidants. Antioxidants can delay the start, or slow the rate, of oxidation and can act by various mechanisms. Some water soluble antioxidants, for example, ascorbic acid and sodium metabisulfite have lower oxidation potentials than the drug, and preferentially undergo degradation. Some oil-soluble anti-oxidants inhibit free radical mediated chain reactions. Examples of such antioxidants include butylated hydroxytoluene, butylated hydroxyanisole, ascorbic esters, tocopherol and lecithin.

In an embodiment, the pharmaceutical formulations of the present invention include one or more antioxidants. Antioxidants for the purposes of the present invention include, but are not limited to, butylated hydroxytoluene, butylated hydroxyanisole, propyl gallate, ascorbic acid palmitate, α-tocopherol, fumaric acid, malic acid, propyl gallate, sodium metabisulfite, derivatives thereof, and the like, including any mixtures thereof.

In an embodiment, an antioxidant is incorporated in the pharmaceutical formulations of the present invention, wherein the antioxidant causes reduction of the peroxide content of the excipient and thereby makes the peroxide unavailable to react with raloxifene to form undesirable amounts of the N-oxide impurity.

In an embodiment, sodium metabisulphite is incorporated in the pharmaceutical formulation of the present invention as a stabilizer which causes reduction of the peroxide content of the excipient and thereby makes the peroxide unavailable to react with the raloxifene.

In an embodiment, pharmaceutical formulations of the present invention are substantially free of the N-oxide impurity. The term “substantially free” of N-oxide impurity means not more than about 5%, or not more than about 3%, or not more than about 0.5%, or not more than about 0.3%, of the N-oxide impurity is present in the formulation, calculated as a percentage of the raloxifene content in the formulation.

In another embodiment, the pharmaceutical formulations of the present invention contain not more than about 2%, or not more than about 1%, by weight of the raloxifene content in the formulation, of total drug-related impurities after storage in closed containers at 40° C. and 75% relative humidity (RH) for a period of at least 2 months.

Pharmaceutical formulations of the present invention may be formulated into different dosage forms such as tablets, capsules, sachets, and like.

Pharmaceutical formulations in addition to surfactants and antioxidants comprise other pharmacologically inactive excipients such as any one or more of diluents, binders, disintegrants, glidants, lubricants, pH modifiers, colouring agents, film coating polymers, and others.

Various useful diluents include but are not limited to starches, lactose, mannitol, Pearlitol™ SD 200, cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, and tribasic calcium phosphate.

Various useful binders include but are not limited to hydroxypropylcelluloses (e.g., Klucel™ LF), hydroxypropyl methylcelluloses or hypromelloses (e.g., Methocel™), polyvinylpyrrolidones or povidones (grades such as PVP-K25, PVP-K29, PVP-K30, and PVP-K90), Plasdone™ S 630 (copovidone), powdered acacia, gelatin, guar gum, carbomers (e.g., Carbopol™) methylcelluloses, polymethacrylates, and starches.

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidones, examples of commercially available crospovidone products including but not limited to crosslinked povidone, Kollidon™ CL (manufactured by BASF, Germany), Polyplasdone™ XL, XI-10, and INF-10 (manufactured by ISP Inc., USA), and low-substituted hydroxypropylcelluloses. Examples of low-substituted hydroxypropylcelluloses include but are not limited to grades such as LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxides, and starches.

Various solvents that can be used in processing include but are not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and any mixtures thereof.

Various glidants or anti-sticking agents can be used, include but are not limited to talc, silica derivatives, colloidal silicon dioxide and the like, and mixtures thereof.

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, zinc stearate, castor oils, and waxes.

Various pH modifiers include but are not limited to various acids such as hydrochloric acid, phosphoric acid, citric acid, carbonic acid, tartaric acid, fumaric acid, acetic acid, etc.; various bases such as sodium hydroxide, magnesium hydroxide, calcium hydroxide, etc.; various salts such as citrates, phosphates, carbonates, tartrates, fumarates, acetates of various alkaline or alkaline earth metals; amino acids and amino acid salts; and meglumine.

Various useful colourants include but are not limited to Food Yellow No. 5, Food Red No. 2, Food Blue No. 2, and the like, food lake colorants, and iron oxides.

In an embodiment, the compositions of the present invention are in the form of coated tablets, or coated multiparticulates filled into a capsule. In an aspect, the compositions are film coated using a coating composition comprising at least one film forming agent.

Various film-forming agents that are useful include but are not limited to cellulose derivatives such as soluble alkyl- or hydroalkyl-cellulose derivatives such as methylcelluloses, hydroxymethylcelluloses, hydroxyethylcelluloses, hydroxypropylcelluloses, hydroxymethyethylcelluloses, hydroxypropyl methylcelluloses (HPMC or hypromelloses, different grades including HPMC 6 cps, HPMC 15 cps, HPMC 50 cps being available), sodium carboxymethylcelluloses, etc., acidic cellulose derivatives such as cellulose acetate phthalates, cellulose acetate trimellitates and methylhydroxypropylcellulose phthalates, polyvinylacetate phthalates, etc., insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acid, polyvinyl alcohols, polyvinyl acetates, polyvinylpyrrolidones, polymethacrylates and derivatives thereof (e.g., Eudragit™ products), chitosan and derivatives thereof, shellac and derivatives thereof, waxes, and fat substances.

If desired, the coating composition may contain additional adjuvants such as plasticizers, polishing agents, colorants, pigments, antifoam agents, opacifiers, anti-sticking agents, and the like, or mixtures thereof.

Various plasticizers include but are not limited to castor oil, diacetylated monoglycerides, dibutyl sebacate, diethyl phthalate, glycerin, polyethylene glycol, propylene glycols, triacetin, and triethyl citrate. Also, mixtures of plasticizers may be utilized. The type of plasticizer depends upon the type of coating agent. A plasticizer is frequently present in an amount ranging from 5% (w/w) to 30 (w/w), based on the total weight of the film coating.

An opacifier like titanium dioxide may also be present in an amount ranging from about 10% (w/w) to about 20% (w/w) based on the total weight of the coating. When coloured tablets are desired then the colour is normally applied in the coating. Consequently, colouring agents and pigments may be present in the film coating. Various colouring agents include but not limited to iron oxides, which can be red, yellow, black or blends thereof.

Anti-adhesives are frequently used in the film coating process to avoid sticking effects during film formation and drying. An example of an anti-adhesive for this purpose is talc. The anti-adhesive typically is present in the film coating in an amount of about 5% (w/w) to 15% (w/w), based upon the total weight of the coating.

Suitable polishing agents include polyethylene glycols of differing molecular weights or mixtures thereof, talc, surfactants (e.g. glycerol mono-stearate and poloxamers), fatty alcohols (e.g. stearyl alcohol, cetyl alcohol, lauryl alcohol and myristyl alcohol) and waxes (e.g. carnauba wax, candelilla wax and white wax). In some embodiments, polyethylene glycols having molecular weights of 3,000-20,000 are employed.

As alternatives for the above coating ingredients, sometimes preformulated coating products such as those sold as OPADRY™ (supplied by Colorcon) will be used, for example Opadry Blue 13B50579 or Opadry™ White 04-58900. These products require only mixing with a liquid before use.

The formulations of the present invention may be prepared by conventional mixing, comminuting and tableting techniques that are well known in the art such as direct compression or dry granulation or wet granulation etc.

Equipment suitable for processing the pharmaceutical formulation of the present invention includes any combination of mechanical sifters, blenders, roller compactors, granulators (rapid mixer or fluid bed granulator) fluid bed dryers, compression machines, rotating bowls or coating pans, etc.

The present invention further relates to processes for manufacturing pharmaceutical formulations of the present invention. In an embodiment of the invention, the process to prepare the formulation comprises a step wherein raloxifene hydrochloride is dispersed in a granulating fluid which is further processed to obtain the desired formulation.

In an embodiment, a formulation manufacturing process comprises:

1) sifting one or more excipients such as diluents, disintegrants, and the like, and optionally active agent, through a sieve and mixing;

2) preparing a dispersion by dispersing active agent in a surfactant solution;

3) granulating the mixture of 1) with the dispersion of 2);

4) drying the granules of 3);

5) separately sifting the dried granules of 4) and extragranular excipients;

6) blending the sifted granules of 5) and extragranular excipients of 5), and adding a lubricant to the blend; and

7) compressing the final lubricated blend of 6) into tablets or filling into capsules.

Tablets or capsules can be subjected to in vitro dissolution evaluations according to Test 711 “Dissolution” in United States Pharmacopoeia 29, United States Pharmacopeial Convention, Inc., Rockville, Md., 2005 (“USP”) to determine the rate at which the active substance is released from the dosage forms, and content of active ingredient can conveniently be determined in solutions by techniques such as high performance liquid chromatography.

The pharmaceutical formulations or dosage forms of the present invention are intended for oral administration to a patient in need thereof.

The present invention also provides methods of using pharmaceutical formulations for preventing and/or treating osteoporosis in a subject in need thereof, such as in postmenopausal women.

In embodiment, the invention includes the use of packaging materials such as containers and closures of high-density polyethylene (HDPE), low-density polyethylene (LDPE) and or polypropylene and/or glass, and blisters or strips composed of aluminium, high-density polypropylene, polyvinyl chloride, polyvinylidine dichloride, etc., into which the formulations are packaged.

Certain specific aspects and embodiments of the invention will be further described in the following examples, which are provided only for purposes of illustration and are not intended to limit the scope of the invention in any manner.

### Example 1

### Raloxifene Hydrochloride 60 mg Tablets

| | |
|---|---|
| Ingredient | Grams |

Drug Dispersion

| | |
|---|---|
| Raloxifene hydrochloride‡ | 165 |
| Poloxamer 407 | 137.5 |
| Methanol-water (80:20 by volume)* | 2750 |

Intragranular

| | |
|---|---|
| Microcrystalline cellulose | 640 |
| Crospovidone | 37.5 |

Extragranular

| | |
|---|---|
| Crospovidone | 162.5 |
| Microcrystalline cellulose | 237.5 |
| Magnesium stearate | 22.5 |

Coating

| | |
|---|---|
| Opadry ™ White 0Y-58900# | 24.75 |
| Water* | 223 |

‡Particle size distribution of raloxifene hydrochloride, as tested using a Horiba Laser Scattering Particle Size distribution analyzer LA-950, are: mean particle size 43.2 μm; and 90% of particles have sizes less than 94 μm.
*Evaporates during processing.
#Opadry White OY 58900 contains HPMC 2910/hypromellose 5 cps, titanium dioxide, and macrogol/PEG 400.

Manufacturing Process:

1) Intragranular microcrystalline cellulose and crospovidone are sifted together through an ASTM #40 mesh sieve and mixed for about 5 minutes, then placed into a fluid bed granulator.

2) Poloxamer is added into the vortex of a stirred methanol-water mixture and then raloxifene hydrochloride is dispersed into it, and stirring is continued for about 5 to 10 minutes until a uniform dispersion forms. The dispersion is passed through an ASTM #120 mesh sieve.

3) Materials of 1) are granulated by a spraying the dispersion of 2) into the fluid bed granulator at an inlet temperature about 55° C. The granules are dried until loss on drying at 105° C. is 2.8% w/w and then are sifted through an ASTM #30 mesh sieve.

4) Extragranular microcrystalline cellulose and crospovidone are sifted through an ASTM #40 mesh sieve and blended with the granules of 3).

5) Magnesium stearate is sifted through an ASTM #80 mesh sieve and blended with the mixture of 4).

6) The blend of 5) is compressed into tablets having an average weight of 550 mg.

7) Opadry White is dispersed in water and stirred for about 45 minutes.

8) Tablets of 6) are coated with Opadry dispersion of 7).

The tablets are tested to determine their in vitro dissolution profile, using USP Type II apparatus in three different media: 0.001 N HCl (900 mL); 0.01 N HCl (1000 mL), pH 4.5 acetate buffer (900 mL); and 0.1% polysorbate 80 in purified water (1000 mL); with a stirring speed of 50 rpm. Values in the table are cumulative percentages of contained drug that dissolve. “T” and “R” represent the product of Example 1 and the commercial product EVISTA 60 mg tablets, respectively.

| | | | |
|---|---|---|---|
| 0.001N | 0.01N | Acetate | Polysor- |
| HCl | HCl | Buffer | bate 80 |

| Minutes | T | R | T | R | T | R | T | R |
|---|---|---|---|---|---|---|---|---|
| 10 | 93 | 74 | 68 | 41 | 84 | 69 | 67 | 72 |
| 20 | 95 | 95 | 75 | 59 | 90 | 92 | 77 | 89 |
| 30 | 96 | 98 | 79 | 67 | 91 | 94 | 83 | 92 |
| 45 | 97 | 99 | 82 | 74 | 92 | 95 | 81 | 92 |

The tablets are evaluated in a two-way crossover pharmacokinetic study using 15 healthy human subjects in the fasting state. The following parameters are calculated:

AUC₀₋ₜ=the area under the drug plasma concentration versus time curve, from time of administration to the time of last measurable concentration.

AUC_{0-∞}=area under the plasma concentration versus time curve, from time of administration to infinity.

Cₘₐₓ=maximum plasma concentration.

Tₘₐₓ=time to the maximum measured plasma concentration.

The pharmacokinetic values are tabulated below:

| | AUC₀₋ₜ | AUC_{0-∞} | Cₘₐₓ | Tₘₐₓ |
|---|---|---|---|---|
| Sample | (pg · hour/ml) | (pg · hour/ml) | (pg/ml) | (hours) |
| Example 1 (“T”) | 14640 | 14876 | 459 | 8 |
| EVISTA | 15342 | 15564 | 471 | 10 |
| 60 mg (“R”) | | | | |
| (T ÷ R) × 100 | 94 | 95 | 104 | — |

### Example 2

### Raloxifene Hydrochloride 60 mg Tablets

| | mg/Tablet | | | |
|---|---|---|---|---|
| Ingredient | A | B | C | D |

Intragranular

| | | | | |
|---|---|---|---|---|
| Raloxifene hydrochloride‡ | 60 | 60 | 60 | 60 |
| Microcrystalline cellulose (RQ102) | 206 | 206 | 206 | 206 |
| Poloxamer 407 | — | 25 | 35 | 50 |
| Methanol* | q.s. | q.s. | q.s. | q.s. |
| Water* | q.s. | q.s. | q.s. | q.s. |

Extragranular

| | | | | |
|---|---|---|---|---|
| Crospovidone | 60 | 60 | 70 | 70 |
| Pregelatinized starch | 45 | 45 | — | — |
| Sodium metabisulfite | — | — | 0.25 | 0.25 |
| Prosolv SMCC90** | 173 | 148 | 157.75 | 157.75 |
| Magnesium stearate | 6 | 6 | 6 | 6 |

Coating

| | | | | |
|---|---|---|---|---|
| Opadry White AMB OY-B-28920# | — | — | 17 | 17 |
| Water* | — | — | q.s. | q.s. |

‡Particle size distribution of unmicronized raloxifene hydrochloride, as tested using a Horiba Laser Scattering Particle Size distribution analyzer LA-950, are: mean particle size 35.54 μm; and 90% of particles have sizes less than 84.67 μm.
*Evaporates during processing.
#Opadry White AMB OY-B-28920 contains polyvinyl acetate, xanthan gum, lecithin, and titanium dioxide.
**Prosolv ® is silicified microcrystalline cellulose, from JRS Pharma.

Manufacturing Process:

1) Microcrystalline cellulose and crospovidone are sifted together through an ASTM #40 mesh sieve and then placed into a fluid bed granulator.

2) Poloxamer 407 is added into the vortex of a stirred methanol-water mixture and then raloxifene hydrochloride is dispersed into it, and stirring is continued for about 5 to 10 minutes until a uniform dispersion is obtained. The size distribution of the particles in the dispersion (as tested using a Malvern Laser Scattering Particle Size distribution analyzer 2000S) are: mean particle size 44.35 μm; and 90% of particles have sizes less than 94.73 μm.

3) Materials of 1) are granulated by a spraying the dispersion of 2) into the fluid bed granulator at an inlet temperature about 55° C. The granules are dried until loss on drying at 105° C. is less than about 3% w/w and then are sifted through an ASTM #30 mesh sieve.

4) The extragranular materials, except magnesium stearate, are sifted through an ASTM #40 mesh sieve and blended with the granules of 3).

5) Magnesium stearate is sifted through an ASTM #60 mesh sieve and blended with the mixture of 4).

6) The blend of 5) is compressed into tablets having an average weight of 550 mg.

7) Opadry White is dispersed in water and stirred for about 45 minutes.

8) Where required, tablets of 6) are coated with Opadry dispersion of 7).

The tablets are tested to determine their in vitro dissolution profiles, using USP Type II apparatus in three different media: 0.01 N HCl (1000 mL); and 0.1% polysorbate 80 in purified water (1000 mL); with a stirring speed of 50 rpm. Values in the table are cumulative percentages of contained drug that dissolve.

| | 0.01N HCl | | | | 0.1% Polysorbate 80 | | | |
|---|---|---|---|---|---|---|---|---|
| Minutes | A | B | C | D | A | B | C | D |
| 10 | 46 | 75 | 65 | 59 | 54 | 59 | 74 | 80 |
| 20 | 55 | 74 | 77 | 76 | 64 | 69 | 78 | 88 |
| 30 | 58 | 75 | 79 | 80 | 68 | 73 | 81 | 90 |
| 45 | 56 | 78 | 81 | 83 | 73 | 74 | 83 | 91 |

Disintegration Times

| | | | | | | | |
|---|---|---|---|---|---|---|---|
| 20 sec. | 4 min. | 4 min. | 7 min. | 20 sec. | 4 min. | 4 min. | 7 min. |

Surprisingly, it is found that the desired dissolution and bioavailability characteristics can be achieved even with unmicronized drug, even when insoluble fillers such as microcrystalline cellulose are used in the formulation.

### Example 3

### Raloxifene Hydrochloride 60 mg Tablets

| | |
|---|---|
| Ingredient | mg/Tablet |

Drug Dispersion

| | |
|---|---|
| Raloxifene hydrochloride | 60 |
| Poloxamer 407 | 50 |
| Water* | q.s. |
| Dimethicone (simethicone) | 0.5 |

Intragranular

| | |
|---|---|
| Microcrystalline cellulose PH102 | 255.5 |
| Crospovidone | 15 |

Extragranular

| | |
|---|---|
| Crospovidone | 65 |
| Microcrystalline cellulose PH102 | 95 |
| Magnesium stearate | 9 |

Coating

| | |
|---|---|
| Opadry ™ White 0Y-58900 # | 17 |
| Water* | q.s. |

*Evaporates during processing.

Manufacturing process: similar to that of Example 1, except that water is used instead of a methanol-water mixture and dimethicone is added in 2) to reduce foam formation, which is observed in the aqueous dispersion.

### Example 4

### Raloxifene Hydrochloride 60 mg Tablets

| Ingredient | Grams |
|---|---|
| Raloxifene hydrochloride | 106.4 |
| Poloxamer 407 | 88.7 |
| Methanol-water (80:20 by volume)* | 1775 |
| Microcrystalline cellulose | 365.2 |
| Crospovidone | 124.1 |
| Sodium metabisulphite | 0.9 |
| Silicified microcrystalline cellulose (Prosolve ™) | 218.1 |
| Magnesium stearate | 10.6 |
| Opadry ™ White 0Y-58900 | 24.8 |
| Isopropyl alcohol* | 144 |
| Water* | 144 |

*Evaporates during processing.
*Prosolv ® is silicified microcrystalline cellulose, from JRS Pharma.

Manufacturing Process:

1) Microcrystalline cellulose (MCC) is sifted through a #40 mesh sieve, loaded into a fluid bed processor and pre-warmed for 10 minutes.

2) Poloxamer is dissolved in a methanol-water mixture and raloxifene hydrochloride is dispersed in the poloxamer solution using a propeller stirrer.

3) The drug dispersion of 2) is sprayed onto the MCC in the fluid bed processor, to form granules.

4) The granules of 3) are dried in the fluid bed processor at an inlet temperature of 60° C. until a loss on drying less than 3% w/w is obtained, then the dried granules are passed through a #20 mesh sieve.

5) Crospovidone, sodium metabisulphite, starch 1500, and silicified microcrystalline cellulose are sifted together through a #40 mesh sieve, and are blended with sifted granules of 4) in a double cone blender.

6) Magnesium stearate is sifted through a #60 mesh sieve and blended with the mixture of 5) in the double cone blender.

7) The blend of 6) is compressed into tablets having an average weight of 564 mg.

8) Opadry White is dispersed in isopropyl alcohol and water and stirred for about 45 minutes.

9) The tablets of 7) are coated with Opadry dispersion of 8).

### Example 5

### Raloxifene Hydrochloride 60 mg Tablets

| Ingredient | Grams |
|---|---|
| Raloxifene hydrochloride | 60 |
| Poloxamer 407 | 50 |
| Methanol-water (80:20 by volume)* | 1000 |
| Microcrystalline cellulose PH102 | 320.5 |
| Crospovidone | 105 |
| Sodium metabisulphite | 0.5 |
| Syloid ™ FP244@ | 5 |
| Magnesium stearate | 9 |
| Opadry ™ White 0Y-58900 # | 15 |
| Isopropyl alcohol | 102 |
| Water* | 102 |

*Evaporates during processing.
@Syloid FP 244 is a silica gel product manufactured by W. R. Grace Corporation of Baltimore, Maryland USA.

Manufacturing process: similar to that of Example 6, except that the Prosolve SMCC is replaced by Syloid FP244.

The tablets are subjected to stability testing with storage at 40° C. and 75% RH, and analysis results are presented in the following table, where values are percentages of the label raloxifene content.

Impurity

| Storage Time | N-Oxide | Total |
|---|---|---|
| Initial | ND* | 0.19 |
| 1 Month | ND | 0.20 |
| 2 Months | ND | 0.17 |

*ND = Not detected.

### Example 6

### Raloxifene Hydrochloride 60 mg Tablets

| Ingredient | Grams |
|---|---|
| Raloxifene hydrochloride | 30 |
| Sodium lauryl sulphate | 25 |
| Hydroxypropyl methylcellulose 5 cps | 5 |
| (HPMC 5 cps) | |
| Microcrystalline cellulose PH101 | 15 |
| Methanol-water (80:20 by volume)* | 55 |
| Microcrystalline cellulose PH102 | 180 |
| Opadry White OY 58900 | 70 |
| Water* | 630 |

*Evaporates during processing.

Manufacturing Process:

1) Raloxifene hydrochloride and sodium lauryl sulphate are sifted together through an ASTM #40 mesh sieve and mixed for about 5 minutes.

2) HPMC 5 cps is dissolved in a methanol-water mixture.

3) The mixture of 1) is granulated using the solution of 2).

4) The granules of 3) are dried until loss on drying is less than 1% at 105° C., and the dried granules are sifted through an ASTM # 20 mesh sieve.

5) The sifted granules of 4) are blended with microcrystalline cellulose.

6) Blend of 5) is compressed into tablets of about 300 mg weight.

7) Opadry White is dispersed in water and stirred for about 45 minutes.

8) The tablets of 6) are coated with Opadry dispersion of 7).

### Example 7

### Raloxifene Hydrochloride 60 mg Tablets

| Ingredient | Grams |
|---|---|
| Raloxifene hydrochloride | 600 |
| Mannitol impalpable | 704 |
| Fumaric acid | 1000 |
| Hydroxypropyl cellulose (Klucel LF) | 70 |
| Polysorbate 80 | 50 |
| Water* | 400 |
| L-hydroxypropyl cellulose (LH-21) | 268.64 |
| Mannitol (Parteck M-200) | 695.11 |
| Colloidal silicon dioxide | 95.7 |
| Talc | 47.85 |
| Zinc stearate | 47.85 |
| Opadry White OY 58900 | 95 |
| Water* | 3166 |

*Evaporates during processing.

Manufacturing Process:

1) Raloxifene hydrochloride and mannitol are sifted together through an ASTM #40 mesh sieve.

2) Fumaric acid is milled through a 0.5 mm screen with knives forward and fast speed, and is sifted through an ASTM #80 mesh sieve.

3) The ingredients of 1) and 2) are sifted together through an ASTM #40 mesh sieve.

4) Material of 3) is transferred into a rapid mixer granulator and dry mixed for about 20 minutes with impeller fast speed and chopper off.

5) Polysorbate 80 is dispersed in water at 60° C. and stirred.

6) Hyrodxypropyl cellulose is added to the solution of 5) and stirred.

7) The mixture of 4) is granulated using the solution of 6).

8) The granules of 7) are dried in a fluid bed dryer at 60±5° C. for about 30 minutes, and then are sifted through an ASTM #25 mesh sieve.

9) L-hydroxypropyl cellulose, colloidal silicon dioxide and mannitol are sifted through an ASTM #30 mesh sieve and blended together with the granules of 8) in a double cone blender

10) Talc and zinc stearate are sifted through an ASTM #100 mesh sieve and mixed with the blend of 9).

11) The lubricated blend of 10) is compressed into tablets having an average weight of 380 mg.

12) Opadry White is dispersed in water and stirred for about 45 minutes.

13) The tablets of 11) are coated with Opadry dispersion of 12).

The tablets are subjected to in vitro dissolution testing with the following dissolution conditions: 0.001 N HCl (900 ml) and 0.1% polysorbate 80 in purified water (900 ml), 50 rpm stirring speed and USP Type II apparatus. The values are cumulative percentages of contained drug that dissolve.

| | 0.1% Polysorbate 80 | | 0.001N HCI | |
|---|---|---|---|---|
| Minutes | Example 7 | EVISTA 60 mg | Example 7 | EVISTA 60 mg |
| 10 | 79 | 75 | 69 | 77 |
| 20 | 90 | 82 | 85 | 94 |
| 30 | 94 | 85 | 91 | 97 |
| 45 | 97 | 83 | 97 | 98 |

### Example 8

### Raloxifene Hydrochloride 60 mg Tablets

| | |
|---|---|
| Ingredient | Grams |

Intragranular

| | |
|---|---|
| Raloxifene hydrochloride | 60 |
| Lactose monohydrate | 219 |
| Crospovidone | 10 |
| Sodium hydroxide | 3 |
| Water* | 40 |

Extragranular

| | |
|---|---|
| Crospovidone | 5 |
| Zinc stearate | 3 |

Film Coating

| | |
|---|---|
| Opadry White OY 58900 | 12.6 |
| Water* | 113.4 |

*Evaporates during processing.

Manufacturing Process:

1) Raloxifene hydrochloride, lactose monohydrate and crospovidone are sifted together through an ASTM #40 mesh sieve and mixed.

2) Sodium hydroxide is dissolved in water.

3) The material of 1) is granulated using the solution of 2).

4) The granules are dried to a loss on drying of about 0.9% w/w.

5) The dried granules of 4) are milled through a 1.5 mm screen and the milled materials are passed through an ASTM #20 mesh sieve.

6) Crospovidone and zinc stearate are sifted through ASTM #40 mesh and #80 mesh sieves, respectively, and blended with the granules of 5) in a double cone blender.

7) The blend of 6) is compressed into tablets of about 300 mg weight.

8) Opadry White is dispersed in water and stirred for about 45 minutes.

9) The tablets of 7) are coated with Opadry dispersion of 9).

### Example 9

### Raloxifene Hydrochloride 60 mg Tablets

| Ingredient | Grams |
|---|---|
| Raloxifene hydrochloride | 30 |
| Beta-cyclodextrin (β-CD) | 30 |
| Water* | 70 |
| Lactose monohydrate impalpable | 117.5 |
| Crospovidone | 5 |
| Crospovidone | 5 |
| Magnesium stearate | 2.5 |

*Evaporates during processing.

Manufacturing Process:

1) Raloxifene hydrochloride and β-CD are sifted together through an ASTM #40 mesh sieve.

2) Lactose monohydrate and crospovidone (first quantity) are sifted together through an ASTM #40 mesh sieve.

3) The mixture of 1) is granulated with half the quantity of water.

4) The blend of 2) and granules of 3) are mixed and granulated with the remaining quantity of water.

5) The granules are dried in a fluid bed dryer until a loss on drying is 1.4% w/w, and the dried granules are sifted through an ASTM #20 mesh sieve.

6) Crospovidone (second quantity) and magnesium stearate are sifted through an ASTM #40 mesh sieve, added to granules of 5), and blended.

7) The lubricated blend of 6) is compressed into tablets with an average weight of 500 mg.

1. A pharmaceutical formulation comprising raloxifene as an active agent, at least one surfactant, and at least one water-insoluble diluent, wherein the active agent has a mean particle size about 30 μm to about 75 μm.

2. A pharmaceutical formulation of claim 1, wherein 90 percent of active agent particles have sizes that do not exceed a size between about 60 μm and about 150 μm.

3. A pharmaceutical formulation of claim 1, wherein raloxifene is in the form of a hydrochloride salt.

4. A pharmaceutical formulation of claim 1, wherein the active agent is in intimate contact with the surfactant.

5. A pharmaceutical formulation of claim 1, wherein the surfactant is a non-ionic surfactant.

6. A pharmaceutical formulation of claim 1, wherein the surfactant comprises a block copolymer of polyethylene oxide) and polypropylene oxide).

7. A pharmaceutical formulation of claim 1, wherein a weight ratio of active agent to surfactant is about 0.3 to about 10.

8. A pharmaceutical formulation of claim 1, wherein a weight ratio of the active agent to the surfactant is about 0.4 to about 5.

9. A pharmaceutical formulation of claim 1, wherein a weight ratio of active agent to surfactant is about 0.5 to about 2.

10. A pharmaceutical formulation of claim 1, wherein a water-insoluble diluent comprises microcrystalline cellulose.

11. A pharmaceutical formulation of claim 1, comprising an antioxidant.

12. A pharmaceutical formulation of claim 11, wherein an antioxidant comprises butylated hydroxytoluene, butylated hydroxyanisole, propyl gallate, ascorbic acid palmitate, alpha-tocopherol, fumaric acid, malic acid, propyl gallate, sodium metabisulfite, or any mixtures thereof.

13. A pharmaceutical formulation of claim 11, wherein an antioxidant comprises sodium metabisulphite.

14. A pharmaceutical formulation of claim 1, comprising at least one disintegrant.

15. A pharmaceutical formulation of claim 14, wherein a disintegrant comprises crospovidone, croscarmellose sodium, or sodium starch glycolate.

16. A pharmaceutical formulation of claim 14, wherein a disintegrant comprises crospovidone.

17. A pharmaceutical formulation of claim 1, containing not more than about 2% by weight of the raloxifene content of total drug-related impurities, after storage in a closed container at 40° C. and 75% relative humidity for a period of at least 2 months.

18. A pharmaceutical formulation of claim 1, containing not more than about 1% by weight of the raloxifene content of total drug-related impurities, after storage in a closed container at 40° C. and 75% relative humidity for a period of at least 2 months.

19. A process for preparing a pharmaceutical formulation of claim 1, comprising one or more of direct compression, dry granulation, wet granulation, and spray granulation.

20. A process for preparing a pharmaceutical formulation of claim 1, comprising: (a) mixing a diluent, a disintegrant, and optionally one or more additional pharmaceutical excipients; (b) granulating the mixture of (a) with a granulating solution or dispersion containing raloxifene; and (c) compressing granules into tablets, optionally together with one or more pharmaceutical excipients.

21. A process for preparing a pharmaceutical formulation of claim 1, comprising: (a) mixing a diluent, a disintegrant, and optionally one or more additional pharmaceutical excipients; (b) granulating the mixture of (a) with a solution or dispersion containing raloxifene; and (c) filling granules into capsules.

22. A method for treating osteoporosis, comprising administering to a patient in need thereof a pharmaceutical formulation of claim 1.

23. A method for treating osteoporosis, comprising administering to a patient in need thereof a pharmaceutical formulation prepared by a process comprising: (a) mixing a diluent, a disintegrant, and optionally one or more additional pharmaceutical excipients; (b) granulating the mixture of (a) with a granulating solution or dispersion containing raloxifene; and (c) compressing granules into tablets, optionally together with one or more pharmaceutical excipients, or filling granules into capsules.
